(19) United States
(12) Patent Application Publication
(10) Pub. No.: US 2026/0334769 A1
(43) Pub. Date: Oct. 29, 2026

(54) ENDOSCOPE REPROCESSOR, METHOD, AND COMPUTER-READABLE RECORDING MEDIUM FOR SUPPLYING FLUID TO CONDUIT OF ENDOSCOPE

(71) Applicant: OLYMPUS MEDICAL SYSTEMS CORP., Tokyo (JP)

(72) Inventor: Ryo SATO, Tokyo (JP)

(73) Assignee: OLYMPUS MEDICAL SYSTEMS CORP., Tokyo (JP)

(21) Appl. No.: 19/569,515

(22) Filed: Mar. 17, 2026

Related U.S. Application Data
(63) Continuation of application No. PCT/JP2023/033985, filed on Sep. 19, 2023.

(51) Int. Cl. A61B 1/12; A61B 1/00

(52) U.S. Cl. CPC A61B 1/123; A61B 1/00055; A61B 1/00128; A61B 1/125

(57) ABSTRACT

An endoscope reprocessor includes a first tube, a second tube, a fluid supply assembly, and a controller. The fluid supply assembly is configured to supply a fluid to a conduit of an endoscope via the first and second tubes. The conduit includes a first conduit communicating with a first ferrule, and a second conduit communicating with a second ferrule, the first and second conduits merging at a merging section into a third conduit having an opening at a distal end of an insertion portion of the endoscope. The controller is configured to control the fluid supply assembly to supply the fluid, while the second ferrule is open, to the second ferrule via the merging section from the first tube connected to the first ferrule and from the second tube connected to a distal end portion of the endoscope.

### CROSS REFERENCE TO RELATED APPLICATION

This application is a continuation of International Application No. PCT/JP2023/033985, filed on Sep. 19, 2023, the entire contents of which are incorporated herein by reference.

### FIELD

The present disclosure relates to an endoscope reprocessor, a method, and a non-transitory computer-readable recording medium for supplying fluid to a conduit of an endoscope.

### BACKGROUND

For endoscopes used in the medical field, reprocessing, such as cleaning and disinfection, is indispensable for reuse after an insertion portion is inserted into a subject to perform observation and treatment with a treatment instrument in the subject.

An endoscope reprocessor removes contaminants such as blood clots and mucus adhering to conduits by connecting a connector of a tube to each of a suction ferrule and a forceps ferrule of the endoscope, and then supplying fluid to each of the tubes to discharge the fluid from an opening at a distal end of the insertion portion.

International Publication No. WO 2015/001843 discloses an endoscope cleaning apparatus that cleans a forceps ferrule using liquid that leaks out from a space between the forceps ferrule and a connector of a tube connected to the forceps ferrule.

International Publication No. WO 2016/194456 discloses an endoscope reprocessor that performs flow control to adjust a flow rate of a first fluid supply unit that supplies liquid as a fluid and a flow rate of a second fluid supply unit that supplies gas as a fluid.

### SUMMARY

According to aspects of the present disclosure, an endoscope reprocessor is provided, which includes a first tube, a second tube, a fluid supply assembly, and a controller. The fluid supply assembly is configured to supply a fluid to a conduit of an endoscope via the first and second tubes. The conduit includes a first conduit communicating with a first ferrule, and a second conduit communicating with a second ferrule, the first and second conduits merging at a merging section into a third conduit having an opening at a distal end of an insertion portion of the endoscope. The controller is configured to control the fluid supply assembly to supply the fluid, while the second ferrule is open, to the second ferrule via the merging section from the first tube connected to the first ferrule and from the second tube connected to a distal end portion of the endoscope.

According to aspects of the present disclosure, further provided is a method implementable by a processor operatively coupled to an endoscope reprocessor including a first tube and a second tube. The method includes operating the endoscope reprocessor to supply a fluid to a conduit of an endoscope via the first and second tubes. The conduit includes a first conduit communicating with a first ferrule, and a second conduit communicating with a second ferrule, the first and second conduits merging at a merging section into a third conduit having an opening at a distal end of an insertion portion of the endoscope. The method further includes controlling the endoscope reprocessor to supply the fluid, while the second ferrule is open, to the second ferrule via the merging section from the first tube connected to the first ferrule and from the second tube connected to a distal end portion of the endoscope.

According to aspects of the present disclosure, further provided is a non-transitory computer-readable recording medium storing computer-readable instructions executable by a processor operatively coupled to an endoscope reprocessor including a first tube and a second tube. The computer-readable instructions are configured to, when executed by the processor, cause the processor to operate the endoscope reprocessor to supply a fluid to a conduit of an endoscope via the first and second tubes. The conduit includes a first conduit communicating with a first ferrule, and a second conduit communicating with a second ferrule, the first and second conduits merging at a merging section into a third conduit having an opening at a distal end of an insertion portion of the endoscope. The computer-readable instructions are further configured to, when executed by the processor, cause the processor to control the endoscope reprocessor to supply the fluid, while the second ferrule is open, to the second ferrule via the merging section from the first tube connected to the first ferrule and from the second tube connected to a distal end portion of the endoscope.

### BRIEF DESCRIPTION OF DRAWINGS

FIG. 1 is a perspective view of an endoscope reprocessor according to a first embodiment.

FIG. 2 is a configuration diagram of a main part of the endoscope reprocessor according to the first embodiment.

FIG. 3 is a configuration diagram of the main part of the endoscope reprocessor according to the first embodiment.

FIG. 4 is a cross-sectional view of a joint section between a connector and a forceps ferrule in the endoscope reprocessor according to the first embodiment.

FIG. 5 is a cross-sectional view of a merging section of conduits in the endoscope reprocessor according to the first embodiment.

FIG. 6 is a view showing a connection configuration between a connector of a second tube of the first embodiment and a distal end portion of an insertion portion.

FIG. 7 is a flowchart describing an example of a flow of cleaning processing for the endoscope reprocessor of the first embodiment.

FIG. 8 is a view showing a state in which an endoscope is placed in a processing tank of the endoscope reprocessor.

FIG. 9 is a view showing a connection configuration between a connector of the second tube of a second embodiment and the distal end portion of the insertion portion.

### DESCRIPTION OF EMBODIMENTS

An endoscope reprocessor 1 of an embodiment of the present disclosure will be described below using the drawings. Hereinafter, the endoscope reprocessor 1 will be referred to as a reprocessor 1.

It should be noted that the drawings based on the embodiment are schematic. The relationship between the thickness and width of each part and the ratio of the thickness of each part differ from the actual ones. The drawings also contain parts in which dimensional relationships and ratios are different from each other.

### First Embodiment

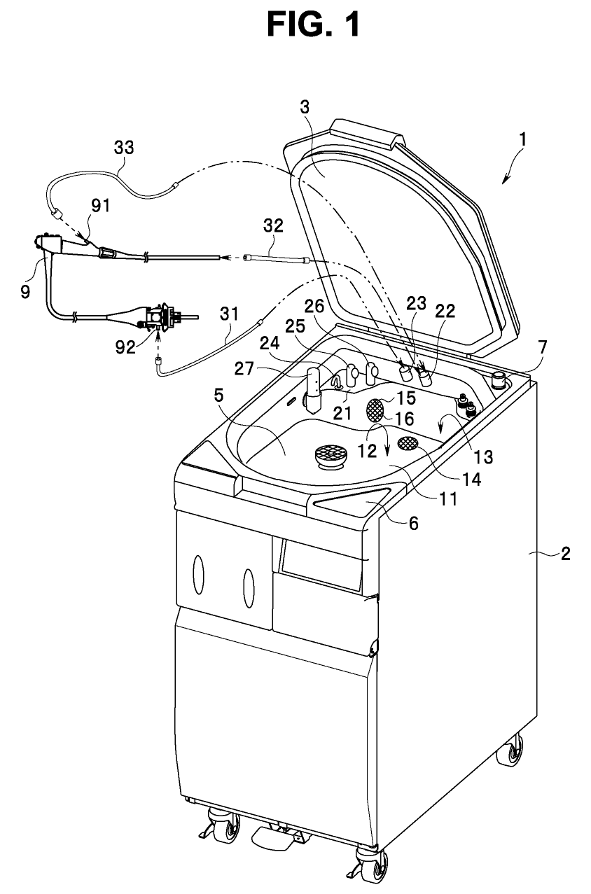

FIG. 1 is a perspective view of an endoscope reprocessor according to a first embodiment. As shown in FIG. 1, the reprocessor 1 includes a main body 2 and a top cover 3 which is openable and closable. FIG. 1 shows a state in which the top cover 3 of the reprocessor 1 is opened.

The reprocessor 1 is an apparatus that performs restoration processing (i.e., reprocessing) of an endoscope 9 or an endoscope accessory. The restoration processing may be rinsing with water, cleaning to clear away contaminants such as organic matter, disinfection to inactivate predetermined microorganisms, sterilization to eliminate or kill all microorganisms, or any combination thereof.

The main body 2 includes, at the top thereof, a processing tank 5 for performing processing such as cleaning and disinfection of the endoscope 9, an operation panel 6, and a water-supply-hose connection port 7.

The processing tank 5 stores a liquid such as a cleaning solution, water, an alcohol disinfectant solution, or a sterilization solution. The processing tank 5 includes an endoscope placement section 11 and a terrace 21.

The endoscope placement section 11 includes a bottom surface 12 and a side surface 13, allows the endoscope 9 to be placed thereon, and stores liquid. A discharge port 14 for discharging the stored liquid is provided in the bottom surface 12 of the endoscope placement section 11. A circulation port 16 having a mesh filter 15 is provided in the side surface 13 of the endoscope placement section 11. The circulation port 16 is in communication with a liquid pump 51 mentioned below. The circulation port 16 may be provided in the bottom surface 12.

The terrace 21 is provided at a position adjacent to the endoscope placement section 11 and higher than the endoscope placement section 11. The terrace 21 includes a forceps plug port 22 which is a second tube connection port, a suction ferrule port 23 which is a first tube connection port, a cleaning solution nozzle 24, a disinfectant solution nozzle 25, a water supply nozzle 26, and a water level sensor 27.

The suction ferrule port 23 is a port to which a first tube 31 is to be connected. The forceps plug port 22 is a port to which a second tube 32 or a third tube 33 is to be connected. The first tube 31, the second tube 32, and the third tube 33 configure a cleaning tube. The number of ports that the reprocessor 1 has is not limited to 2. For example, the reprocessor 1 may include three ports to which the first tube 31, the second tube 32, and the third tube 33 are to be connected, respectively.

The cleaning solution nozzle 24 supplies a cleaning solution to the processing tank 5. The disinfectant solution nozzle 25 supplies a disinfectant solution to the processing tank 5. The water supply nozzle 26 supplies water taken in from the water-supply-hose connection port 7 to the processing tank 5, and also supplies the liquid in the processing tank 5 taken in from the circulation port 16 having the mesh filter 15 back to the processing tank 5, thereby circulating the liquid. The mesh filter 15 filters contaminants P from the liquid. The water level sensor 27 detects a water level of the liquid stored in the processing tank 5.

The operation panel 6 is placed in a front section on top of the reprocessor 1. The operation panel 6 includes various operation buttons, not shown. A user gives various instructions to the reprocessor 1 through the operation panel 6.

The water-supply-hose connection port 7 is provided at a rear section on top of the reprocessor 1. A water supply hose connected to an unshown water faucet is connected to the water-supply-hose connection port 7, and water is supplied to the reprocessor 1 via the water supply nozzle 26.

The top cover 3 is provided openably and closably on the top of the main body 2. In the reprocessor 1, by bringing the top cover 3 into an open state, the endoscope 9 can be placed on the endoscope placement section 11, and the endoscope 9 and the reprocessor 1 can be connected by the first tube 31 and the second tube 32, or the first tube 31 and the third tube 33. The reprocessor 1 is in a state in which processing such as cleaning and disinfection is possible by bringing the top cover 3 into a closed state after the endoscope 9 is set.

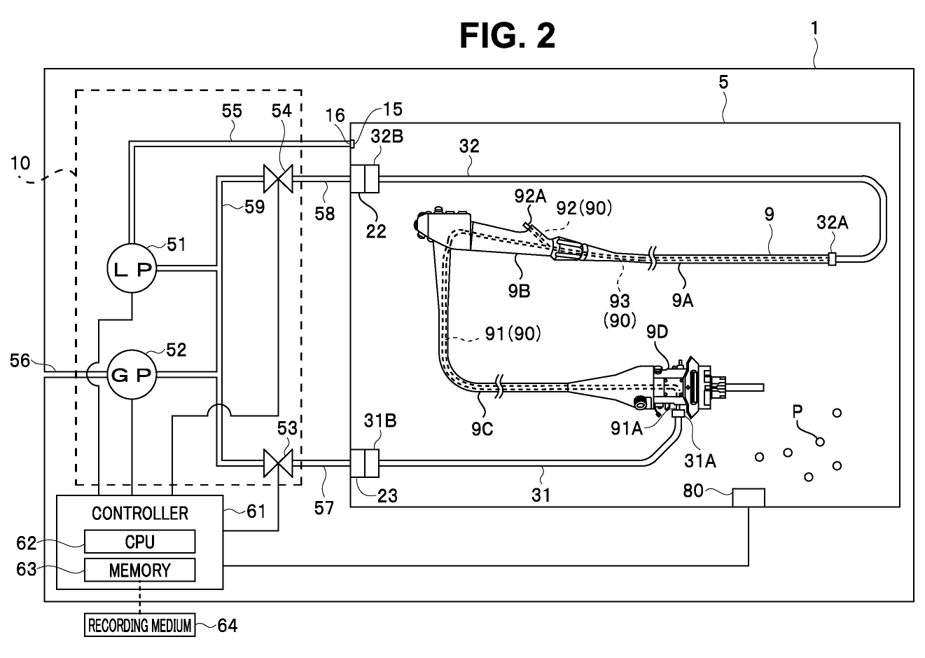

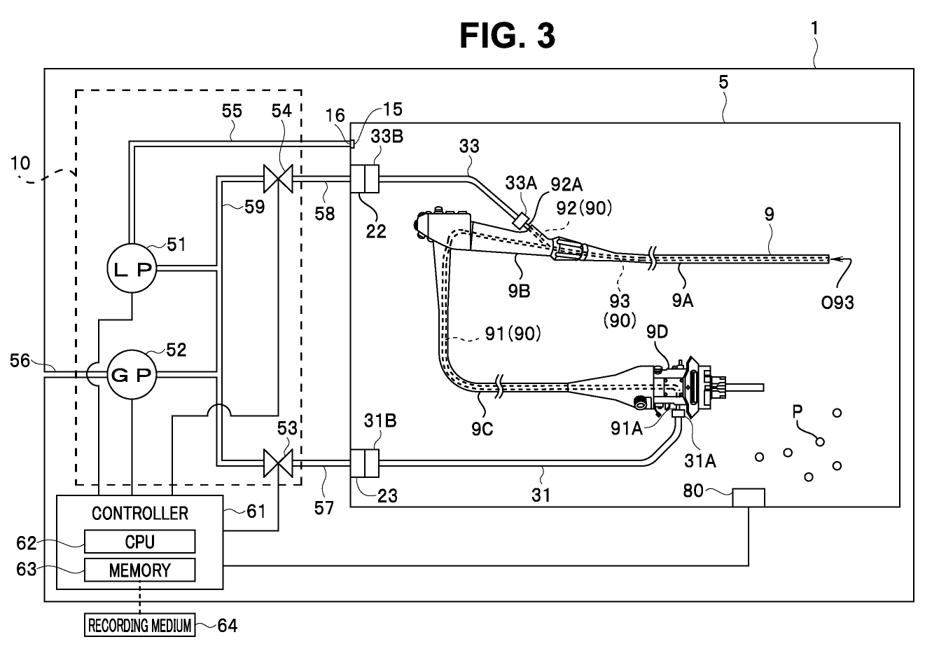

FIGS. 2 and 3 are each a configuration diagram of a main part of the endoscope reprocessor according to the first embodiment. FIG. 2 shows a state in which the endoscope 9 to which the first tube 31 and the second tube 32 are connected is housed in the reprocessor 1. FIG. 3 shows a state in which the endoscope 9 to which the first tube 31 and the third tube 33 are connected is housed in the reprocessor 1. It should be noted that FIGS. 2 and 3 show only the main configuration in the present disclosure. The reprocessor 1 may have a configuration different from the configurations shown in FIGS. 2 and 3 as long as it has the same function as those shown in FIGS. 2 and 3.

The endoscope 9 includes an insertion portion 9A to be inserted into a body, an operation portion 9B, a universal cord 9C, and an endoscope connector 9D. The endoscope 9 includes a conduit 90 inside. The conduit 90 includes a first conduit 91, a second conduit 92, and a third conduit 93. A suction ferrule 91A is provided at one end of the first conduit 91, and the other end thereof merges with the second conduit 92. A forceps ferrule 92A is provided at one end of the second conduit 92, and the other end thereof merges with the first conduit 91.

The third conduit 93, where the first conduit 91 and the second conduit 92 merge, passes through the insertion portion 9A and has an opening O93 at a distal end (see FIG. 3). A treatment instrument, such as a forceps, inserted from the forceps ferrule 92A passes through the second conduit 92 and the third conduit 93, and its distal end protrudes from the opening O93. Fluid fed from the suction ferrule 91A is emitted from the opening O93 via the first conduit 91 and the third conduit 93.

The first tube 31 includes a connector 31A provided at one end thereof and connected to the suction ferrule 91A, which is a first ferrule of the endoscope 9, and a connector 31B provided at the other end thereof and connected to the suction ferrule port 23 of the reprocessor 1. The suction ferrule port 23 is in communication with the suction ferrule 91A of the endoscope 9 via the first tube 31.

The second tube 32 includes a connector 32A provided at one end thereof and connected to a distal end portion of the insertion portion 9A of the endoscope 9, and a connector 32B provided at the other end thereof and connected to the forceps plug port 22 of the reprocessor 1. Upon connection of the second tube 32, the forceps plug port 22 is in communication with the distal end portion of the insertion portion 9A of the endoscope 9 via the second tube 32.

The third tube 33 shown in FIG. 3 includes a connector 33A provided at one end thereof and connected to the forceps ferrule 92A, which is a second ferrule of the endoscope 9, and a connector 33B provided at the other end thereof and connected to the forceps plug port 22 of the reprocessor 1. Upon connection of the third tube 33, the forceps plug port 22 is in communication with the forceps ferrule 92A of the endoscope 9 via the third tube 33.

The reprocessor 1 includes the liquid pump 51, a gas pump 52, the processing tank 5, a first solenoid valve 53, a second solenoid valve 54, the forceps plug port 22, the suction ferrule port 23, and a controller 61.

The liquid pump 51 takes in liquid, such as cleaning solution in the processing tank 5, from the circulation port 16 having the mesh filter 15 into a conduit 55, pressurizes the taken-in liquid at a predetermined pressure, and feeds the pressurized liquid to a branch conduit 59. Although not shown, a part of the liquid taken in from the circulation port 16 is fed to the processing tank 5 by another pump. The mesh filter 15 filters the contaminants P that flow down from the conduit 90 of the endoscope 9 and float in the liquid in the processing tank 5.

The liquid pump 51 is connected to the first solenoid valve 53 and the second solenoid valve 54 via the branch conduit 59. The first solenoid valve 53 is connected to the suction ferrule port 23 via a conduit 57. The second solenoid valve 54 is connected to the forceps plug port 22 via a conduit 58.

The gas pump 52 takes in gas via a conduit 56, pressurizes the taken-in gas at a predetermined pressure, and feeds the pressurized gas to the branch conduit 59. The gas is air, for example. The gas pump 52 is connected to the first solenoid valve 53 and the second solenoid valve 54 via the branch conduit 59.

The processing tank 5 is provided with a sensor 80 that detects contaminants in the liquid stored in the tank 5. The sensor 80 includes a turbidity sensor or the like configured to measure a turbidity of the liquid. The turbidity sensor detects the contaminants P in the liquid by measuring light transmittance of the liquid. Since the stored liquid circulates in the conduit 90, when the contaminants P in the conduit 90 are removed, the contaminants P in the stored liquid are also reduced. In other words, the contaminants P detected by the sensor 80 can be regarded as contaminants P in the conduit 90. The sensor 80 may preferably be placed in the vicinity of the forceps ferrule 92A of the endoscope 9 placed in the processing tank 5.

The controller 61 may be implemented by processing circuitry including one or more processors. For instance, the controller 61 includes processing circuitry including a CPU 62, which is a central processing unit of a computer, and a memory 63 including a ROM, a RAM, or the like. Functions of the controller 61 are realized by the CPU 62 reading a program from the memory 63 and executing the read program. A program for causing the computer to execute reprocessing which is stored in the memory 63 is retained in a non-transitory computer-readable recording medium 64, and may be transferred to the memory 63.

The controller 61 is electrically connected to the liquid pump 51, the gas pump 52, the first solenoid valve 53, the second solenoid valve 54, and the sensor 80. The controller 61 may be implemented as a separate control apparatus operatively coupled to the reprocessor 1 in a wired or wireless manner. In this case, the controller 61 and the reprocessor 1 may form an endoscope reprocessing system.

When supplying liquid to the conduit 90, the controller 61 activates the liquid pump 51 and stops the gas pump 52. When supplying gas to the conduit 90, the controller 61 stops the liquid pump 51 and activates the gas pump 52.

When supplying a gas-liquid two-phase flow to the conduit 90, the controller 61 activates the liquid pump 51 and then activates the gas pump 52. The gas-liquid two-phase flow refers to a state in which liquid and gas are present in a mixed state in the conduit 90 by feeding air from the gas pump 52 in a state in which the conduit 90 of the endoscope 9 is filled with liquid. More specifically, the gas-liquid two-phase flow includes any of the following states: a state in which gas bubbles are present in liquid, a state in which liquid droplets are present in gas, and a state in which liquid slugs and gas pockets are present adjacent to one another.

The controller 61 controls an opening or closing state of the first solenoid valve 53, so that a fluid at a predetermined flow rate (predetermined pressure) is supplied to the first conduit 91 via the first tube 31. When the forceps plug port 22 and the distal end portion of the insertion portion 9A are connected by the second tube 32, the controller 61 controls an opening or closing state of the second solenoid valve 54, so that a fluid at a predetermined flow rate is supplied to the third conduit 93 via the second tube 32. When the forceps plug port 22 and the forceps ferrule 92A are connected by the third tube 33, the controller 61 controls an opening or closing state of the second solenoid valve 54, so that a fluid at a predetermined flow rate is supplied to the second conduit 92 via the third tube 33.

The liquid pump 51, the gas pump 52, the first solenoid valve 53, and the second solenoid valve 54 configure a fluid supply unit (hereinafter may be referred to as a fluid supply assembly) 10. The controller 61 controls timing at which the fluid supply unit 10 supplies fluid to the conduit 90.

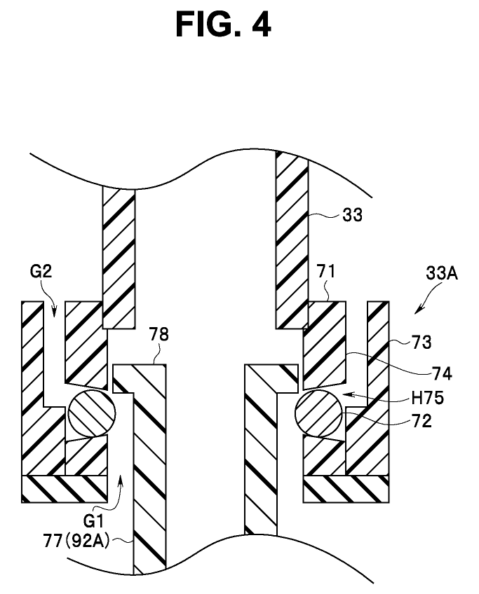

FIG. 4 is a cross-sectional view of a joint section between a connector in the endoscope reprocessor and a forceps ferrule according to the first embodiment.

The connector 33A includes a connector main body 71 provided at a distal end of the third tube 33, a plurality of spheres 72, and a connector cover 73 provided on an outer circumferential section of the connector main body 71.

The connector main body 71 is configured of plastic or the like. The connector main body 71 is tubular, and includes a plurality of circular holes H75 in a circumferential side portion 74. For example, four holes H75 are provided in the circumferential side portion 74 of the connector main body 71 at equal intervals along a circumferential direction. Each of the holes H75 has a decreasing diameter from an outer surface toward an inner surface of the connector main body 71, and has a tapered cross section in a thickness direction of the circumferential side portion 74.

The plurality of spheres 72 is configured of metal or the like. The plurality of spheres 72 each has a diameter larger than that of each of the holes H75 in an inner circumferential surface of the circumferential side portion 74, and is placed with respective parts fitted in the holes H75, so that the spheres do not fall off from the inner circumferential surface of the circumferential side portion 74.

The connector cover 73 is configured of plastic or the like. The connector cover 73 is placed externally to the connector main body 71 where the plurality of spheres 72 is placed. A circumferential gap G2 is formed between the connector cover 73 and the connector main body 71.

The forceps ferrule 92A is made of metal, resin, or the like. The forceps ferrule 92A has a body section 77 formed in a cylindrical shape, and includes an outwardly extending flange 78 at a distal end thereof.

The connector 33A of the third tube 33 is detachably attached to the forceps ferrule 92A. In a state in which the connector 33A is attached to the forceps ferrule 92A, the plurality (four, in this case) of spheres 72 of the connector 33A engages with the outwardly extending flange 78 of the forceps ferrule 92A to inhibit detachment from the flange. A circumferential gap G1 is formed between the body section 77 of the forceps ferrule 92A and the connector main body 71.

The fluid fed from the third tube 33 is introduced to an inner side of the body section 77 of the forceps ferrule 92A, and flows out from the gap G2 via the space between the connector main body 71 in which the hole H75 is formed and the sphere 72, and also flows out from the gap G1 via the space between the connector main body 71 and the forceps ferrule 92A. The fluid flowing out from the gaps G1 and G2 in a connection region between the connector 33A and the forceps ferrule 92A cleans the forceps ferrule 92A.

It should be noted that the shape of the connector 33A of the third tube 33 is not limited to that mentioned above, and a connector disclosed in International Publication No. WO 2015/001843 already described can also be employed.

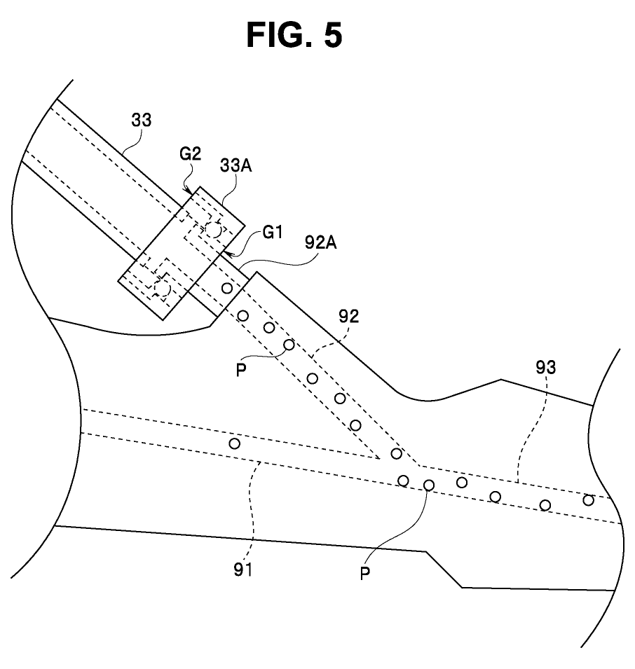

FIG. 5 is a cross-sectional view of a merging section of conduits in the endoscope reprocessor according to the first embodiment. As shown in FIG. 5, the contaminants P may adhere to the conduit 90 of the endoscope 9 after use, particularly to the second conduit 92 and the third conduit 93 which are withdrawal paths for the treatment instrument inserted into the body.

As already described, since there are the gaps G1 and G2 between the connector 33A of the third tube 33 connected to the forceps ferrule 92A and the forceps ferrule 92A, in the reprocessing, when the fluid supplied to the first conduit 91 flows back through the second conduit 92 via the merging section, the contaminants P flushed away by the fluid may be trapped in the gaps G1 and G2.

In general, since the insertion portion 9A to be inserted into the body has a small diameter, the third conduit 93 provided within the insertion portion 9A also has a smaller diameter than the first conduit 91 and the second conduit 92. For this reason, the conduit resistance in the third conduit 93 within the insertion portion 9A is larger than the conduit resistance in the first conduit 91 and the conduit resistance in the second conduit 92. As a result, a part of the fluid fed from the suction ferrule 91A does not flow to the distal end side of the insertion portion 9A, but flows back to the forceps ferrule 92A side, causing the contaminants P to be trapped in the gaps G1 and G2.

In the present embodiment, first, liquid is fed from the suction ferrule 91A and the distal end portion of the insertion portion 9A in a state in which the second tube 32 is connected to the distal end portion of the insertion portion 9A, and the third tube 33 is not connected to the forceps ferrule 92A and the forceps ferrule 92A is left open. This causes the contaminants P adhering to the second conduit 92 and the third conduit 93, which are the withdrawal paths for the treatment instrument, to be discharged from the forceps ferrule 92A.

Thereafter, in place of the second tube 32, the third tube 33 is connected to the forceps ferrule 92A, and liquid is fed from the suction ferrule 91A and the forceps ferrule 92A. This inhibits the contaminants P from being trapped in the forceps ferrule 92A.

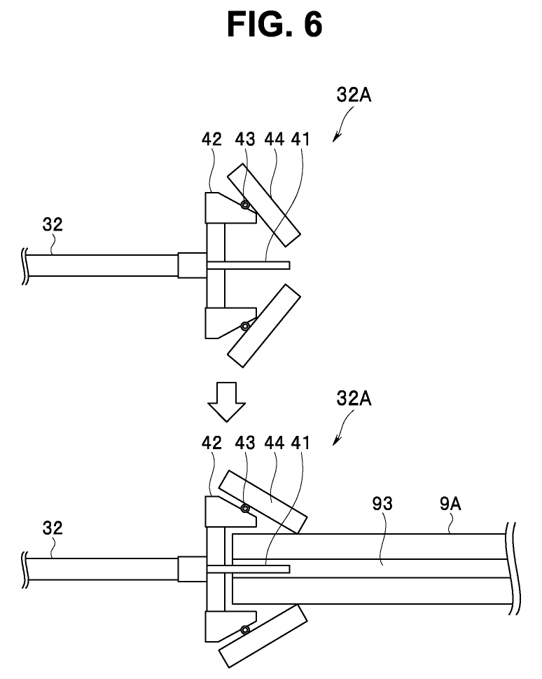

FIG. 6 is a view showing a connection configuration between a connector of the second tube of the first embodiment and the distal end portion of the insertion portion.

The connector 32A of the second tube 32 includes a nozzle 41 for feeding liquid to the third conduit 93, an insertion member 42 into which the distal end portion of the insertion portion 9A is to be inserted, a spring 43 for clamping the insertion portion 9A, and a fixing member 44 for clamping and fixing the insertion portion 9A with the force of the spring 43.

The nozzle 41 is put into the third conduit 93 within the insertion portion 9A, the insertion portion 9A is clamped by means of the spring 43 and the fixing member 44, and the second tube 32 is fixed to the distal end portion of the insertion portion 9A so as not to be detached during liquid feeding. In this manner, the second tube 32 is fixed to the distal end portion of the insertion portion 9A, and liquid is fed from the nozzle 41 of the second tube 32 to the third conduit 93.

Next, cleaning processing as an action of the endoscope reprocessor 1 will be described.
The user opens the top cover 3 of the endoscope reprocessor 1, and sets the endoscope 9 to be cleaned in the endoscope reprocessor 1. Specifically, the user connects the connector 31B of the first tube 31 to the suction ferrule port 23, and connects the connector 31A to the suction ferrule 91A of the endoscope 9. The user also connects the connector 32B of the second tube 32 to the forceps plug port 22, and connects the connector 32A to the distal end portion of the insertion portion 9A of the endoscope 9. The forceps ferrule 92A is left in an open state. It should be noted that, although not shown, in addition to the connection by the first tube 31 and the second tube 32, the endoscope reprocessor 1 and the endoscope 9 are connected by tubes as necessary.

After connecting the endoscope reprocessor 1 and the endoscope 9, the user places the endoscope 9 in the endoscope placement section 11, and brings the top cover 3 into the closed state.

When the user gives a start instruction for processing such as cleaning and disinfection from the operation panel 6, the CPU 62 reads a predetermined program from a memory 63 and starts processing the program.

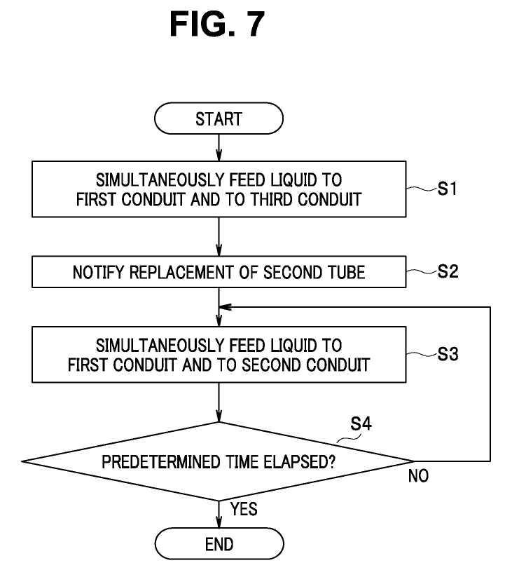

FIG. 7 is a flowchart describing an example of a flow of cleaning processing for the endoscope reprocessor of the first embodiment.

The controller 61 drives the fluid supply unit 10 to simultaneously feed liquid to the first conduit 91 and to the third conduit 93 (S1). By this processing, the fed liquid is discharged from the forceps ferrule 92A which is in the open state, so that the contaminants P adhering to the second conduit 92 and the third conduit 93 and the contaminants P adhering in the vicinity of the forceps ferrule 92A are discharged from the forceps ferrule 92A.

It should be noted that the controller 61 may alternately feed liquid to the first conduit 91 and to the third conduit 93. When alternately feeding liquid to the first conduit 91 and to the third conduit 93, the controller 61 increases the liquid feeding rate or feeds liquid at a high pressure. The controller 61 starts feeding liquid from the suction ferrule 91A or the distal end portion of the insertion portion 9A, and after a fixed time has elapsed, stops feeding liquid from the suction ferrule 91A or the distal end portion of the insertion portion 9A. Thereafter, the controller 61 starts feeding liquid from the suction ferrule 91A or the distal end portion of the insertion portion 9A from which liquid has not been fed. The timing of stopping and starting feeding liquid is a timing when the contaminants P do not reach from one of the suction ferrule 91A or the distal end portion of the insertion portion 9A to the other. For example, the controller 61 controls the timing of stopping and starting feeding liquid by calculating a flow velocity of the fluid passing through the suction ferrule 91A and flowing into the conduit 90 and a velocity of the fluid passing through the distal end portion of the insertion portion 9A and flowing in the conduit 90.

Subsequently, the controller 61 notifies replacement of the second tube 32 (S2). In other words, in S2, the controller 61 issues a notification to replace the second tube 32. When the turbidity of the liquid (e.g., water) detected by the sensor 80 becomes less than a predetermined value, the controller 61 determines that the contaminants P in the conduit 90 including the second conduit 92 have been substantially removed. Then, the controller 61 stops feeding liquid to the first conduit 91 and to the third conduit 93, and generates an instruction signal for notifying the user. The instruction signal is a signal instructing to detach the second tube 32 from the forceps plug port 22 and the distal end portion of the insertion portion 9A, and connect the third tube 33 to the forceps plug port 22 and the forceps ferrule 92A. The instruction signal is provided, for example, as at least one of a visual indication (e.g., a display on the operation panel 6) or an audible indication (e.g., a sound from a speaker).

When notified of replacement of the second tube 32, the user replaces the second tube 32. Specifically, the user opens the top cover 3 of the endoscope reprocessor 1, and detaches the second tube 32 from the endoscope reprocessor 1 and the endoscope 9. Then, the user connects the connector 33B of the third tube 33 to the forceps plug port 22, and connects the connector 33A to the forceps ferrule 92A of the endoscope 9. The distal end portion of the insertion portion 9A is left in an open state. After connecting the endoscope reprocessor 1 and the endoscope 9 by the third tube 33, the user places the endoscope 9 on the endoscope placement section 11, and brings the top cover 3 into the closed state.

The controller 61 drives the fluid supply unit 10 to simultaneously feed liquid to the first conduit 91 and to the second conduit 92 (S3). It should be noted that the controller 61 may alternately feed liquid to the first conduit 91 and to the second conduit 92. The controller 61 starts feeding liquid from the suction ferrule 91A or the forceps ferrule 92A, and after a fixed time has elapsed, stops feeding liquid from the suction ferrule 91A or the forceps ferrule 92A. Thereafter, the controller 61 starts feeding liquid from the suction ferrule 91A or the forceps ferrule 92A from which liquid has not been fed. The timing of stopping and starting feeding liquid is a timing at which the contaminants P do not reach from one of the suction ferrule 91A or the forceps ferrule 92A to the other.

The fact that the third conduit 93 has a high conduit resistance is a cause for backflow to the forceps ferrule 92A. In order to inhibit the backflow to the forceps ferrule 92A, the conduit resistance in the third conduit 93 may be lowered by exposing, during liquid feeding, the distal end portion of the insertion portion 9A to the atmosphere from the processing tank 5 in which liquid is stored.

Then, the controller 61 determines whether or not a predetermined time has elapsed (S4). If determining that the predetermined time has not elapsed (S4: NO), the controller 61 continues the cleaning processing in S3. On the other hand, if determining that the predetermined time has elapsed (S4: YES), the controller 61 ends the cleaning processing.

In this manner, before a step of cleaning processing of feeding liquid from the suction ferrule 91A and the forceps ferrule 92A, a step of not connecting the third tube 33 to the forceps ferrule 92A is provided. In other words, the forceps ferrule 92A is left in an open state, and liquid is fed from the suction ferrule 91A and the distal end portion of the insertion portion 9A, thereby discharging the contaminants P in the second conduit 92 and the third conduit 93 from the forceps ferrule 92A.

This makes it possible to inhibit the space between the forceps ferrule 92A and the connector 33A of the third tube 33 from being clogged with the contaminants P in the step of cleaning processing of feeding liquid from the suction ferrule 91A and the forceps ferrule 92A.

### Second Embodiment

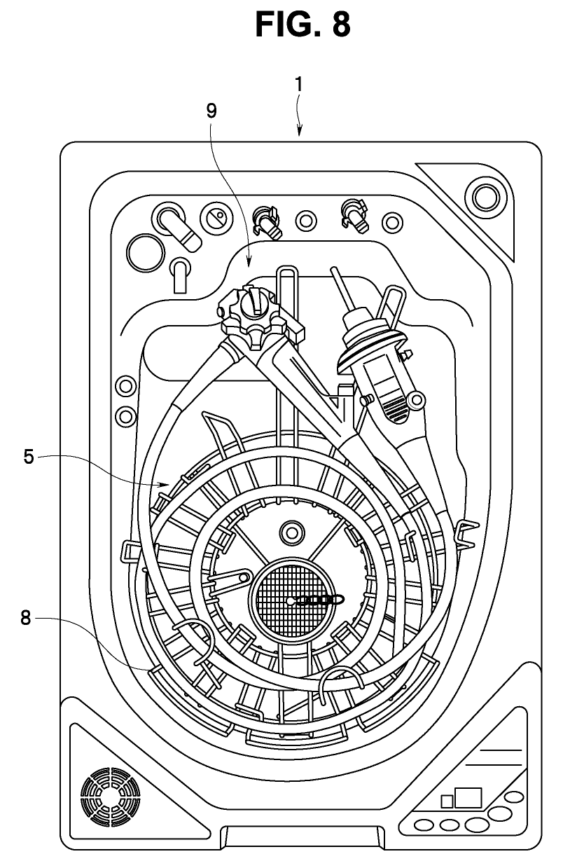

Next, a second embodiment will be described.
FIG. 8 is a view showing a state in which the endoscope is placed in the processing tank of the endoscope reprocessor.
As shown in FIG. 8, the endoscope 9 is placed within the processing tank 5 of the reprocessor 1. A holding net 8 formed of metal or resin, for example, is attached to a bottom section of the processing tank 5. When reprocessing is performed, the endoscope 9 is placed on the holding net 8 within the processing tank 5.

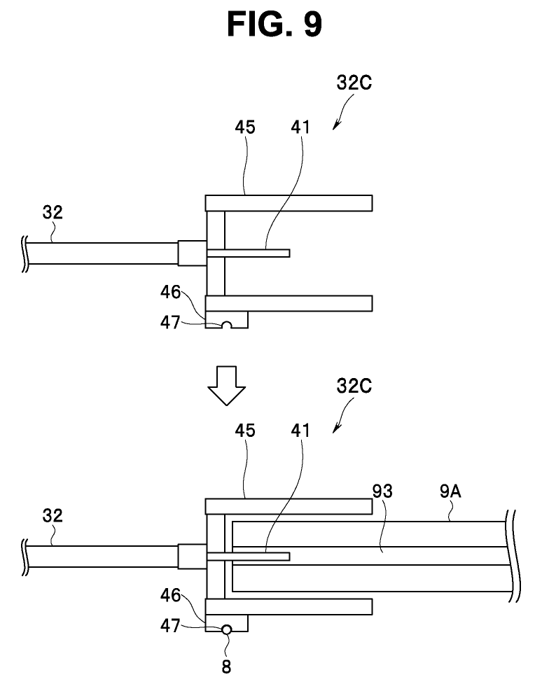

FIG. 9 is a view showing a connection configuration between a connector of the second tube of a second embodiment and the distal end portion of the insertion portion.
The second tube 32 includes a connector 32C instead of the connector 32A in FIG. 6. The connector 32C of the second tube 32 includes a nozzle 41 for feeding liquid to the third conduit, an insertion member 45 into which the distal end portion of the insertion portion 9A is inserted, and a fixing member 46 for fixing the second tube 32 to the holding net 8.

The insertion member 45 has a predetermined length to inhibit the insertion portion 9A from pulling out (coming out or slipping out) upon insertion thereof. As shown in FIG. 8, the insertion portion 9A of the endoscope 9 is placed in a coiled manner on the holding net 8 in the processing tank 5. For this reason, when the insertion member 45 has a certain length, when the distal end portion of the insertion portion 9A is curved, an outer surface of the distal end portion comes into contact with an inner surface of the insertion member 45 thus inhibiting the insertion portion 9A from being pulled out.

The fixing member 46 includes a groove 47 into which the holding net 8 is to be fitted. After attaching the connector 32C to the distal end portion of the insertion portion 9A, the user fits the holding net 8 into the groove 47 of the fixing member 46 to fix the net.

In this manner, by fixing the connector 32C to the holding net 8, it is possible to inhibit the second tube 32 and the insertion portion 9A from shifting in position during liquid feeding.

It should be noted that the configuration of the connector to be connected to the distal end portion of the insertion portion 9A is not limited to that of the connector 32A shown in FIG. 6 or the connector 32C shown in FIG. 9, but may be other configurations. For example, a configuration may be employed in which the connector 32A shown in FIG. 6 includes the fixing member 46 shown in FIG. 9 for fixing the second tube 32 to the holding net 8.

The steps in the flowchart in the present specification may, unless contrary to the nature thereof, be executed in a changed order, executed simultaneously, or executed in a different order for each execution.

The present disclosure is not limited to the above-mentioned embodiments, and it is needless to say that various modifications, combinations, and applications are possible without departing from the gist of the disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another. Similarly, the term “non-transitory computer-readable (storage) medium” encompasses a single storage medium or a group of multiple storage media, which may be locally or remotely distributed and may collectively store and provide access to instructions, data, or other information in a coordinated or distributed manner.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:

Clause 1: An endoscope reprocessor comprising:
a first tube configured to be connected to a first ferrule of an endoscope;
a second tube configured to be connected to a distal end portion of the endoscope;
a fluid supply unit configured to supply fluid to a conduit of the endoscope via each of the first tube and the second tube; and
a controller configured to control the fluid supply unit, wherein
the conduit includes a first conduit in communication with the first ferrule, a second conduit in communication with a second ferrule of the endoscope, and a third conduit in communication with a merging section where the first conduit and the second conduit merge, the third conduit including an opening at a distal end of an insertion portion of the endoscope, and
the controller controls the fluid supply unit so as to, when the second ferrule is in an open state, supply the fluid from the first tube and the second tube to the second ferrule via the merging section.

Clause 2: The endoscope reprocessor according to clause 1, further comprising:
a sensor configured to detect contaminants in the conduit, wherein
when the contaminants detected by the sensor are less than a predetermined value, the controller outputs an instruction signal instructing to detach the second tube from the distal end portion and connect the third tube to the second ferrule.

Clause 3: The endoscope reprocessor according to clause 2, wherein the sensor is a turbidity sensor or an optical sensor configured to measure a turbidity of the fluid passing through the conduit.

Clause 4: The endoscope reprocessor according to clause 2, wherein the instruction signal is a display or a sound for notifying the user.

Clause 5: The endoscope reprocessor according to clause 1, wherein the controller is configured to calculate a velocity of the fluid passing through the first ferrule and flowing in the conduit of the endoscope, and a velocity of the fluid passing through the distal end portion of the endoscope and flowing in the conduit of the endoscope, and to control the fluid supply unit so as to supply the fluid, based on the calculated velocities, at a timing when the fluid does not reach from the distal end portion to the first ferrule, or from the first ferrule to the distal end portion.

Clause 6: The endoscope reprocessor according to clause 1, wherein the controller is configured to control the fluid supply unit so as to supply the fluid from either the first ferrule or the distal end portion, and after a predetermined time elapses, to start supplying the fluid from another of the first ferrule and the distal end portion from which the fluid has not been supplied.

Clause 7: The endoscope reprocessor according to clause 1, wherein the second tube includes a nozzle for feeding liquid to the conduit of the endoscope, and when the second tube is mounted to the distal end portion, the distal end portion is fixed to the second tube.

Clause 8: The endoscope reprocessor according to clause 7, wherein the second tube includes a fixation member for fixing the second tube to a holding net on which the endoscope is to be placed.

Clause 9: A method of operating an endoscope reprocessor for an endoscope, the endoscope comprising:
a first conduit in communication with a first ferrule;
a second conduit in communication with a second ferrule; and
a third conduit in communication with a merging section where the first conduit and the second conduit merge, the third conduit including an opening at a distal end of an insertion portion of the endoscope, and
the method comprising controlling so as to, when the second ferrule is in an open state, supply a fluid from a first tube and a second tube to the second ferrule via a merging section, the first tube being configured to be connected to the first ferrule, and the second tube being configured to be connected to a distal end portion of the endoscope.

Clause 10: The method of operating the endoscope reprocessor according to clause 9, the method further comprising:
when contaminants in a conduit of the endoscope detected by the sensor are less than a predetermined value, outputting an instruction signal instructing to detach the second tube from a distal end portion of the endoscope and connect the third tube to the second ferrule.

Clause 11: The method of operating the endoscope reprocessor according to clause 10, wherein the sensor is a turbidity sensor or an optical sensor configured to measure a turbidity of the fluid passing through the conduit.

Clause 12: The method of operating the endoscope reprocessor according to clause 10, wherein the instruction signal is a display or a sound for notifying the user.

Clause 13: The method of operating the endoscope reprocessor according to clause 9, the method further comprising:
controlling so as to calculate a velocity of the fluid passing through the first ferrule and flowing in the conduit of the endoscope, and a velocity of the fluid passing through the distal end portion of the endoscope and flowing in the conduit of the endoscope, and to supply the fluid, based on the calculated velocities, at a timing when the fluid does not reach from the distal end portion to the first ferrule, or from the first ferrule to the distal end portion.

Clause 14: The method of operating the endoscope reprocessor according to clause 9, the method further comprising:
controlling so as to supply the fluid from either the first ferrule or a distal end portion, and after a predetermined time elapses, to start supplying the fluid from another of the first ferrule and the distal end portion from which the fluid has not been supplied.

Clause 15: The method of operating the endoscope reprocessor according to clause 9, wherein the second tube includes a nozzle for feeding liquid to the conduit of the endoscope, and when the second tube is mounted to a distal end portion, the distal end portion is fixed to the second tube.

Clause 16: The method of operating the endoscope reprocessor according to clause 15, wherein the second tube includes a fixation member for fixing the second tube to a holding net on which the endoscope is to be placed.

Clause 17: A non-transitory computer-readable recording medium having recorded thereon a computer program for reprocessing an endoscope, the endoscope comprising:
a first conduit in communication with a first ferrule;
a second conduit in communication with a second ferrule; and
a third conduit where the first conduit and the second conduit merge, the third conduit including an opening at a distal end of an insertion portion of the endoscope, and
the computer program being configured to, when the second ferrule is in an open state, supply a fluid from a first tube and a second tube to the second ferrule via a merging section, the first tube being configured to be connected to the first ferrule, and the second tube being configured to be connected to a distal end portion of the endoscope.

Clause 18: The recording medium according to clause 17, the recording medium having recorded thereon a computer program configured to, when contaminants in a conduit of the endoscope detected by the sensor are less than a predetermined value, output an instruction signal instructing to detach the second tube from a distal end portion of the endoscope and connect the third tube to the second ferrule.

Clause 19: The recording medium according to clause 18, wherein the sensor is a turbidity sensor or an optical sensor configured to measure a turbidity of the fluid passing through the conduit.

Clause 20: The recording medium according to clause 18, wherein the instruction signal is a display or a sound for notifying the user.

What is claimed is:

1. An endoscope reprocessor comprising:
a first tube;
a second tube;
a fluid supply assembly configured to supply a fluid to a conduit of an endoscope via the first and second tubes, the conduit including a first conduit communicating with a first ferrule, and a second conduit communicating with a second ferrule, the first and second conduits merging at a merging section into a third conduit having an opening at a distal end of an insertion portion of the endoscope; and
a controller configured to control the fluid supply assembly to supply the fluid, while the second ferrule is open, to the second ferrule via the merging section from the first tube connected to the first ferrule and from the second tube connected to a distal end portion of the endoscope.

2. The endoscope reprocessor according to claim 1, further comprising a sensor configured to detect a contamination level in the conduit,
wherein the controller is further configured to output an instruction signal to prompt removal of the second tube from the distal end portion and connection of a third tube to the second ferrule when the contamination level detected by the sensor is less than a predetermined value.

3. The endoscope reprocessor according to claim 2, wherein the sensor includes a turbidity sensor or an optical sensor configured to measure a turbidity of the fluid having passed through the conduit.

4. The endoscope reprocessor according to claim 2, wherein the instruction signal is provided as at least one of a visual indication or an audible indication for user notification.

5. The endoscope reprocessor according to claim 1, wherein the controller is further configured to:
calculate a velocity of the fluid passing through the first ferrule and flowing in the conduit, and a velocity of the fluid passing through the distal end portion of the endoscope and flowing in the conduit; and
based on the calculated velocities, control the fluid supply assembly to supply the fluid at a timing such that the fluid supplied from one of the first ferrule or the distal end portion does not reach the other.

6. The endoscope reprocessor according to claim 1,
wherein the controller is further configured to control the fluid supply assembly to:
supply the fluid from one of the first ferrule or the distal end portion; and
after a lapse of a predetermined time, start supplying the fluid from the other of the first ferrule or the distal end portion.

7. The endoscope reprocessor according to claim 1, wherein the second tube includes a nozzle for feeding the fluid to the conduit of the endoscope, and is configured to be fixed to the distal end portion when attached thereto.

8. The endoscope reprocessor according to claim 7, wherein the second tube includes a fixing member configured to fix the second tube to a holding net on which the endoscope is placed.

9. A method implementable by a processor operatively coupled to an endoscope reprocessor comprising a first tube and a second tube, the method comprising:
operating the endoscope reprocessor to supply a fluid to a conduit of an endoscope via the first and second tubes, the conduit including a first conduit communicating with a first ferrule, and a second conduit communicating with a second ferrule, the first and second conduits merging at a merging section into a third conduit having an opening at a distal end of an insertion portion of the endoscope; and
controlling the endoscope reprocessor to supply the fluid, while the second ferrule is open, to the second ferrule via the merging section from the first tube connected to the first ferrule and from the second tube connected to a distal end portion of the endoscope.

10. The method according to claim 9, further comprising:
receiving a detection result from a sensor configured to detect a contamination level in the conduit; and
outputting an instruction signal to prompt removal of the second tube from the distal end portion of the endoscope and connection of a third tube to the second ferrule when the contamination level detected by the sensor is less than a predetermined value.

11. The method according to claim 10, wherein the sensor includes a turbidity sensor or an optical sensor configured to measure a turbidity of the fluid having passed through the conduit.

12. The method according to claim 10, wherein the instruction signal is provided as at least one of a visual indication or an audible indication for user notification.

13. The method according to claim 9, wherein the controlling includes:
calculating a velocity of the fluid passing through the first ferrule and flowing in the conduit, and a velocity of the fluid passing through the distal end portion of the endoscope and flowing in the conduit; and
based on the calculated velocities, supplying the fluid at a timing such that the fluid supplied from one of the first ferrule or the distal end portion does not reach the other.

14. The method according to claim 9, wherein the controlling includes:
supplying the fluid from one of the first ferrule or the distal end portion; and
after a lapse of a predetermined time, starting to supply the fluid from the other of the first ferrule or the distal end portion.

15. The method according to claim 9, wherein the second tube includes a nozzle for supplying the fluid to the conduit of the endoscope, and is configured to be fixed to the distal end portion when attached thereto.

16. The method according to claim 15, wherein the second tube includes a fixing member configured to fix the second tube to a holding net on which the endoscope is placed.

17. A non-transitory computer-readable recording medium storing computer-readable instructions executable by a processor operatively coupled to an endoscope reprocessor comprising a first tube and a second tube, the computer-readable instructions being configured to, when executed by the processor, cause the processor to at least perform:
operating the endoscope reprocessor to supply a fluid to a conduit of an endoscope via the first and second tubes, the conduit including a first conduit communicating with a first ferrule, and a second conduit communicating with a second ferrule, the first and second conduits merging at a merging section into a third conduit having an opening at a distal end of an insertion portion of the endoscope; and
controlling the endoscope reprocessor to supply the fluid, while the second ferrule is open, to the second ferrule via the merging section from the first tube connected to the first ferrule and from the second tube connected to a distal end portion of the endoscope.

18. The non-transitory computer-readable recording medium according to claim 17, wherein the computer-readable instructions are further configured to, when executed by the processor, cause the processor to at least further perform:
receiving a detection result from a sensor configured to detect a contamination level in the conduit; and
outputting an instruction signal to prompt removal of the second tube from the distal end portion of the endoscope and connection of a third tube to the second ferrule when the contamination level detected by the sensor is less than a predetermined value.

19. The non-transitory computer-readable recording medium according to claim 18, wherein the sensor includes a turbidity sensor or an optical sensor configured to measure a turbidity of the fluid having passed through the conduit.

20. The non-transitory computer-readable recording medium according to claim 18, wherein the instruction signal is provided as at least one of a visual indication or an audible indication for user notification.
